(19) United States
(12) Patent Application Publication
(10) Pub. No.: US 2026/0272598 A1
(43) Pub. Date: Sep. 17, 2026

(54) MODULAR IMPLANT DELIVERY AND POSITIONING SYSTEM

(71) Applicant: lotaMotion, Inc., lowa City, IA (US)

(72) Inventors: Adam Hahn, Pittsburgh, PA (US); Parker Reineke, Cedar Rapids, IA (US)

(21) Appl. No.: 19/480,281

(22) PCT Filed: May 1, 2024
(86) PCT No.: PCT/US2024/027268

§ 371 Date: Oct. 30, 2025

Related U.S. Application Data
(60) Provisional application No. 63/463,428, filed on May 2, 2023.

(51) Int. Cl. A61B 90/11; A61B 34/30; A61N 1/05; A61N 1/36

(52) U.S. Cl. CPC A61B 90/11; A61B 34/30; A61N 1/0541; A61N 1/36038

(57) ABSTRACT

This document discusses, among other things, systems, and methods for robotically assisted implantation of an implant in a patient. A system includes an implant-positioning unit configured to engage an elongate member of the implant, and a drive unit include a motor. The drive unit is coupled to the implant-positioning unit via a flexible driveshaft. The implant-positioning unit and the drive unit can be held in a housingthat allows for handheld use of the system. The system can be used to robotically control the delivery and positing of a cochlear implant during a hearing-preservation cochlear implant surgery.

### CLAIM OF PRIORITY

This patent application claims the benefit of priority to U.S. Provisional Application Ser. No. 63/463,428, filed May 2, 2023, which is incorporated by reference herein in its entirety.

### TECHNICAL FIELD

This document relates generally to medical systems and more particularly to systems, devices, and methods for robotic control of delivery, positioning, and manipulation of an implant, such as a cochlear implant (also referred to as a cochlear electrode).

### BACKGROUND

The cochlea is the auditory portion of the inner ear. It comprises a spiraled, hollow, conical chamber of bone in which sound waves propagate from the base to the apex of the cochlea. The sound waves vibrate the perilymph that moves hair cells in the organ of Corti, converting the vibrations to electrical signals that are sent to the cochlear nerve. The hair cells and nerves in the basal or outer region of the spiraled cochlea are more sensitive to higher frequencies of sound and are frequently the first part of the cochlea to lose sensitivity. The apical or inner region of the spiraled cochlea is more sensitive to lower frequencies.

Moderate to profound hearing loss affects a large amount of people worldwide and can have a significant impact on a patient physical and mental health, education, employment, and overall quality of life. Hearing loss can be caused by partial damage to the cochlea. Many patients with various degrees of hearing loss have partial damage to the cochlea in the high-frequency regions (basal cochlea) from common causes such as noise exposure, drugs, genetic mutations, or aging, but can retain adequate low-frequency hearing.

Cochlear implants have been used to treat patients with hearing loss. A cochlear implant is a medical device that comprises an external sound processor, a subcutaneously implantable stimulator, and an electrode assembly sized and shaped for cochlear insertion. The sound processor can convert sound signals into electrical signals and transmit the electrical signals to the implantable stimulator. Based on the physical properties (e.g., frequencies) of the received electrical signals, the stimulator can generate electrical impulses to stimulate specific regions in the cochlea via an array of electrodes on the electrode assembly surgically inserted into the cochlea. The region for stimulation can be determined based on the frequencies of the received electrical signals. For example, higher frequencies can result in stimulation at the outer or basal cochlear region, and lower frequencies can result in stimulation at the inner or apical cochlear region.

For patients who have lost high-frequency hearing and consequently have significant difficulty with word understanding but who have substantial residual, low-frequency hearing function in apical cochlea, a short electrode assembly can be indicated to electrically stimulate the basal or outer cochlea to restore high-frequency hearing. A cochlear implant surgery can be performed by a surgeon to manually insert the electrode assembly into the damaged portion of a patient cochlea (e.g., basal cochlea), while avoiding or minimizing any trauma to the undamaged cochlear regions to preserve the low-frequency hearing function. The cochlear implant can be used together with a hearing aid that acoustically stimulates the undamaged low-frequency sensitive apical cochlea.

Intracochlear trauma can occur from large pressure spikes generated during the insertion of cochlear implant electrodes. Cochlear implant surgery can also involve insertion of a guide sheath or tube near or partially into the cochlea. Insertion of any solid or flexible bodies, tubes, or sheaths into the cochlea could elicit similar fluid and force spikes. These pressures spikes can be of sufficient intensity to cause trauma similar to that of an acoustic blast injury and are one likely source for postoperative loss of residual hearing. Similar to the insertion trauma cause by electrode insertion, the manual insertion of a sheath or other solid body/tube manually into the cochlea can cause intracochlear fluid pressure spikes and result in intracochlear damage.

Cochlear implant surgery is just one of a wide variety of medical procedures that require precise insertion and positioning of an electrode within the human body. Many of the challenges encountered with implantation of a cochlear electrode are also challenges in other similar procedures. Accordingly, the following disclosure is also applicable to a variety of procedures involving electrodes or similar medical devices.

### SUMMARY

A hearing-preservation cochlear implant surgery involves implanting an electrode assembly into the damaged cochlear region, while avoiding any trauma to the undamaged cochlear region to preserve any normal residual hearing. In current cochlear implant surgery, a surgeon manually inserts an electrode assembly into patient cochlea. However, a complete manual maneuvering of the electrode assembly can cause undesirable outcome in some patients. For example, manual insertion of electrode assembly can lack precision in implant position and motion control, such as the control of insertion rate, distance, or forces applied to the implant for advancing the electrode assembly to the target cochlear region. This can cause damage to fragile cochlear structures such as local trauma to cochlea wall and hair cells, and result in residual hearing loss.

Complete manual maneuvering of the electrode assembly can also be subject to high inter-operator variability among surgeons. The inter-operator variability is demonstrated in dramatic differences in patient outcomes between institutions and surgeons of differing skill levels. Some patients undergoing hearing-preservation cochlear implant surgery can experience additional hearing decline weeks to years after surgery. Such a continual decline in hearing function can be attributed to an inflammatory response to the trauma inflicted during an initial cochlear implant surgery. Some clinical studies show that techniques aimed at reducing electrode-insertion forces during surgery have improved patient hearing preservation outcomes. For at least reasons, the present inventors have recognized that there remains a need to improve patient outcome following a hearing-preservation cochlear implant surgery, particularly systems, apparatus, and methods that enhance surgical precision in implant delivery and positioning and reduce the risk of perioperative trauma to undamaged cochlea region.

This document discusses, among other things, systems, devices, and methods for robotically assisted implantation of an implant in a patient, such as for delivering and positioning a cochlear implant for treating hearing loss in a hearing-preservation cochlear implant surgery. The systems and devices discussed can also be adapted for robotically controlling insertion of a guide sheath or tube that can be used in conjunction with electrode implantation in a wide variety of medical procedures. The modular system discussed herein includes an external positioning unit reversibly interfacing with and securely engaging an implant such as a cochlear implant having an elongate member, and a computerized control unit for robotically controlling the external positioning unit to regulate the motion of the implant. The computerized control unit can have a user interface that enables a user (e.g., a surgeon) to program various motion control parameters or to select an implantation protocol. The system can include sensors providing feedback on the position or the motion of the implant, or the force or friction applied to the implant during the implantation procedure. The computerized control unit can regulate the motion of the implant based on user input and the sensor feedback. The control systems can also interface with external systems providing electrophysiological measurements to enable closed loop feedback on electrode positioning in real-time during implantation.

In an example, a system for robotically assisted manipulation of an implant, the system comprising: a drive head configured to engage an elongate member of the implant and robotically deliver and position the implant into a target implantation site, the drive head including: a housing including a first half and a second half, the first half coupled to the second half with a hinge, to enable the housing to receive the elongate member; a guide track configured to accommodate and channel the elongate member in a linear motion; and a coupling unit including a wheel, the coupling unit configured to translate the elongate member using the wheel within the guide track, in response to a motion control signal; a power system electrically coupled to the coupling unit, the power system including a motor and motor control circuitry positioned within the housing and configured to drive the wheel of the coupling unit; and a control console communicatively coupled to the power system, the control console including a controller circuit configured to generate the motion control signal, to robotically deliver and position the implant into the target implantation site. In an example, one of the first half or the second half can be a split half including a top half drive housing and a bottom half drive housing. The split half housing allows for an elongate implant/member to be threaded into an implant channel created by the non-split half and the bottom half drive housing.

The present systems and apparatus can improve the use of implants with different shapes or profiles. For example, some cochlear implants (and electrode arrays thereof) can have a curved section to allow the implant to better follow a patient cochlea path to reduce insertion trauma more effectively to the surrounding tissue or the implant, and to bring electrode contacts closer to target stimulation nerve. According to various embodiments discussed in this document, the present systems and apparatus include a modular robotic drive head that features to improve use with electrodes or elongate.

The modular design of the robotically assisted implantation system, as discussed in this document, allows for easy replacement or interchange of a particular module. This can not only improve the system reusability and efficiency but can also reduce the cost of system maintenance. For example, the external positioning unit can be a single-use device positioned in a sterile surgical field or in contact with the patient during an implantation surgery and is disposable after surgery. The computerized control unit can be positioned in a non-sterile field, such as a control room, and can be reused with interchangeable external positioning units.

The external positioning unit is a non-implanted external device. Compared to a partially or completely implantable insertion device, the external positioning unit discussed herein can substantially reduce the risk of complications associated with surgical implantation, extraction, or replacement of otherwise partially or completely implantable insertion device. The external positioning unit also has the advantage of easy troubleshooting, maintenance, and replacement, thereby reducing cost of the system and the procedure. As to be discussed in the following, the external positioning unit can have a small size with limited mechanical and electrical parts, thus making it flexible for external fixation to a patient.

Although the discussion in this document focuses on cochlear implant, this is meant only by way of example and not limitation. It is within the contemplation of the present inventors, and within the scope of this document, that the systems, devices, and methods discussed herein can be configured for robotically delivering, steering, positioning, or extracting various types of implants or prosthesis. By way of non-limiting examples, the implants can include leads, catheter, guidewire, or other mechanical or electrical devices. The implants can be designed for temporary or permanent implantation. The implants can be used for medical diagnosis of a disease or other conditions such as diagnostic catheters, or for therapeutic purposes of cure, mitigation, treatment, or prevention of disease, such as implantable electrodes for stimulating cardiac, neural, muscular, or other tissues. In addition to new implantation, the systems, devices, and methods discussed herein can also be used to surgically reposition or replace an existing implant.

Additional details on a number of exemplary embodiments of the base implant positioning unit can be found in U.S. application Ser. No. 16/926,335, titled “Modular Implant Delivery and Positioning System”, filed Jul. 10, 2020, now issued as U.S. Pat. No. 10,945,761, the entire contents of which is hereby incorporated by reference.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, a sideview of an embodiment of an implant-positioning unit for engaging an elongate member of an implant.

FIGS. 2A-2D illustrate, by way of example and not limitation, various views of an implant drive head with a split drive housing.

FIGS. 3A-3B illustrate, by way of example and not limitation, a locking mechanism for a lower portion of a split drive housing.

FIG. 4 illustrates, by way of example and not limitation, a perspective view of an implant drive head with an open split drive housing including an upper locking mechanism.

FIG. 5 illustrates, by way of example and not limitation, a rear perspective view of an implant drive head with a split drive housing including an upper spring loaded hinge.

FIGS. 6A-6D illustrate, by way of example and not limitation, various view of an implant-positioning unit in a handheld attachment.

FIGS. 7A-7E illustrate, by way of example and not limitation, geared primary and secondary drive wheels within an implant drive head.

FIGS. 8A-8E illustrate, by way of example and not limitation, a brake mechanism for a lower drive housing in a split drive housing of an implant drive head.

### DETAILED DESCRIPTION

In the following detailed description, reference is made to the accompanying drawings which form a part hereof, and in which is shown by way of illustration specific embodiments in which the invention can be practiced. These embodiments are described in sufficient detail to enable those skilled in the art to practice the invention, and it is to be understood that the embodiments can be combined, or that other embodiments can be utilized, and that structural, logical, and electrical changes can be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

Disclosed herein are systems, devices, and methods for robotically assisted implantation of an implant in a patient. Examples of the implants can include leads, catheter, guidewire, guide sheath, or other mechanical or electrical devices. The implants can be designed for temporary or permanent implantation. The implants can additionally be used for medical diagnosis of a disease or other conditions such as diagnostic catheters, or for therapeutic purposes of cure, mitigation, treatment, or prevention of disease, such as implantable electrodes for stimulating cardiac, neural, muscular, or other tissues. The present system can be implemented using a combination of hardware and software designed to provide precise control of implant movement, such as insertion of a cochlear implant during a hearing-preservation cochlear implant surgery, or positioning or manipulation of a cochlear implant in a thyroplasty surgery. The system includes an implant-positioning unit a control console communicatively coupled to the implant-positioning unit. The implant-positioning unit includes a drive head configured to engage an elongate member of the implant and robotically deliver and position the implant into a target implantation site. The control console can have a user interface that enables a user to input motion control instructions. The control console can generate a motion control signal, according to a specific motion control instruction, to control the external positioning unit to propel the implant into a target implant site.

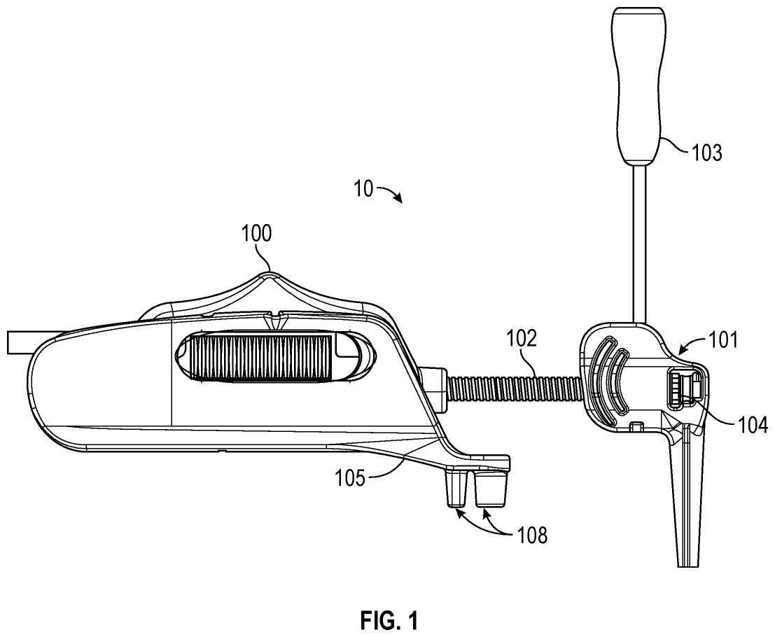

FIG. 1 illustrates, by way of example and not limitation, a sideview of an embodiment of an implant-positioning unit for engaging an elongate member of an implant. The implant-positioning unit 10 in FIG. 1 is an embodiment of a device configured to robotically deliver and position an implant (e.g., a cochlear implant, a thyroplasty implant, or similar electrode-based implant) attached to the elongate member into a target site, or to manipulate soft tissue therein, such as a cochlear or a vocal cord. In an example, the elongate member includes a flexible or semi-flexible elongate implant adapted to position a series of electrodes within the cochlea of a patient (e.g., a cochlear implant or cochlear electrode).

The implant-positioning unit 10 can include a power system contained in a separate housing (e.g., drive unit 100) from the drive head 101 (e.g., wheels including a drive wheel 104 and secondary wheel (discussed below) arrangement) for engaging the elongate member. In this example, the drive unit 100 (power system) can include a motor and motor control circuitry that provide driving force to the coupling unit via an adjustable gooseneck 102. In other examples, the drive unit 100 can transmit rotational forces to the drive head 101 via a flexible drive shaft or similar construct. A flexible drive shaft can include one or more flexible mechanical structures suitable to transmit rotational forces in a bi-directional manner (bi-directional capabilities allow for moving an implant in two directions with the drive head). As illustrated, the implant-positioning unit includes a slidable drive unit 100, and an implant drive head 101 (drive head). The drive unit 100 sits within a stage 105 that can be sized and shaped to conform to the anatomical surface, and can include anchoring members 108 (e.g., self-tapping, captive bone screw holes) that secure drive unit 100 to an anatomical structure to stabilize the implant-positioning unit 10. In some examples, the stage 105 can be C-shaped to hold and compress the drive unit 100 therein. This allows linear movement and increased travel range of the drive unit 100 and drive head 101 for optimal positioning of the drive head 101 and varying anatomical sizes.

In this example, enclosed within the drive unit 100 includes an electric motor that can generate driving force and motion, and a motor controller (e.g., a printed circuit board) that can generate motion control signal to control movement of the electric motor. The electric motor and the motor controller can be respectively connected to a power source and an external control computer via a power/communication cord, such as a USB cable. The electric motor could also be battery-powered, with wireless communication between external control computer and motor controller implemented over proprietary or standard protocols, such as Bluetooth or TCP/IP over 802.11 protocols.

The implant drive head 101 can be connected to the drive unit 100 via an adjustable arm, such as an adjustable gooseneck arm 102. The adjustable arm 102 can be a flexible, semirigid arm that allows for multiple degrees-of-freedom (DOF) adjustment of the drive head 101 at multiple, different angles to the insertion implant site, providing adjustable stability of the drive head 101. In an example, the adjustable arm 102 can be bended to adjust the position of the implant drive head 101. This allows for easy advancement of the elongate member and the associated implant into the target site (e.g., cochlea or vocal cord).

In the example illustrated in FIG. 1, the implant-positing system (unit) 10 can also include a trajectory tool 103. The trajectory tool 103 can assist a practitioner with aligning the drive head 101 along a desired trajectory for insertion of an implant. The trajectory tool 103 is discussed in additional detail below.

### Trajectory Tool

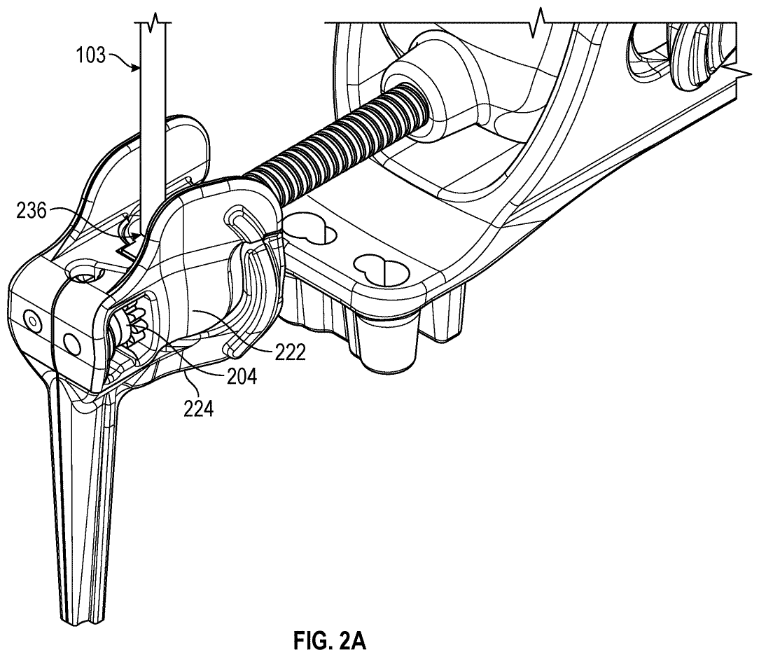

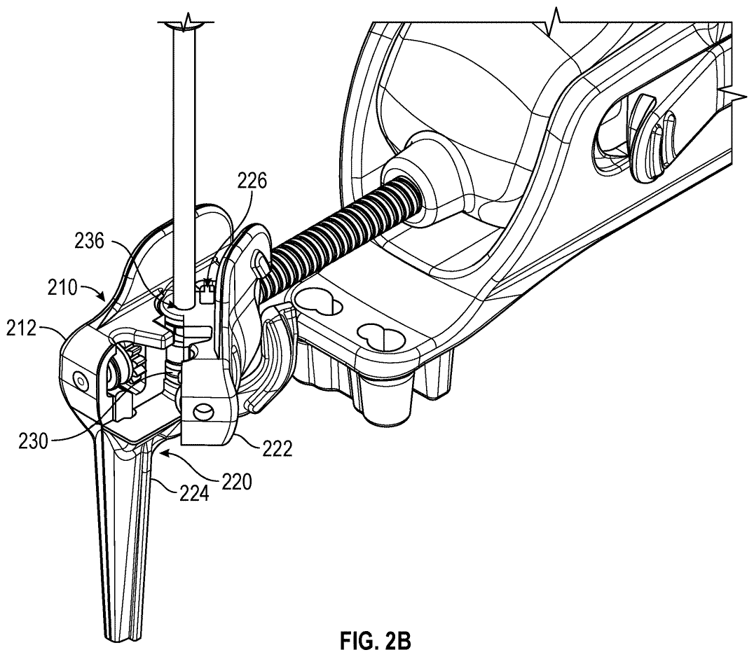

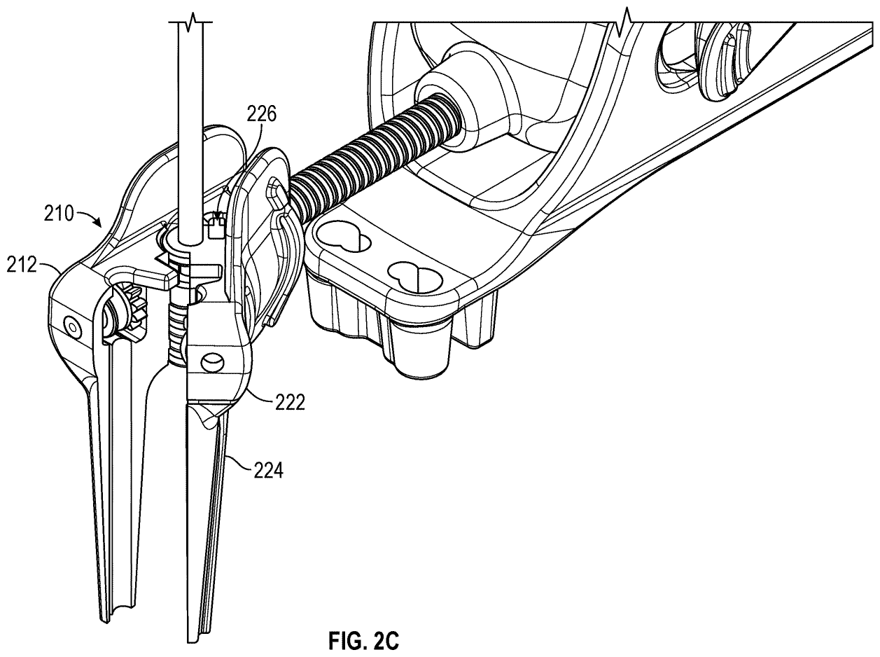

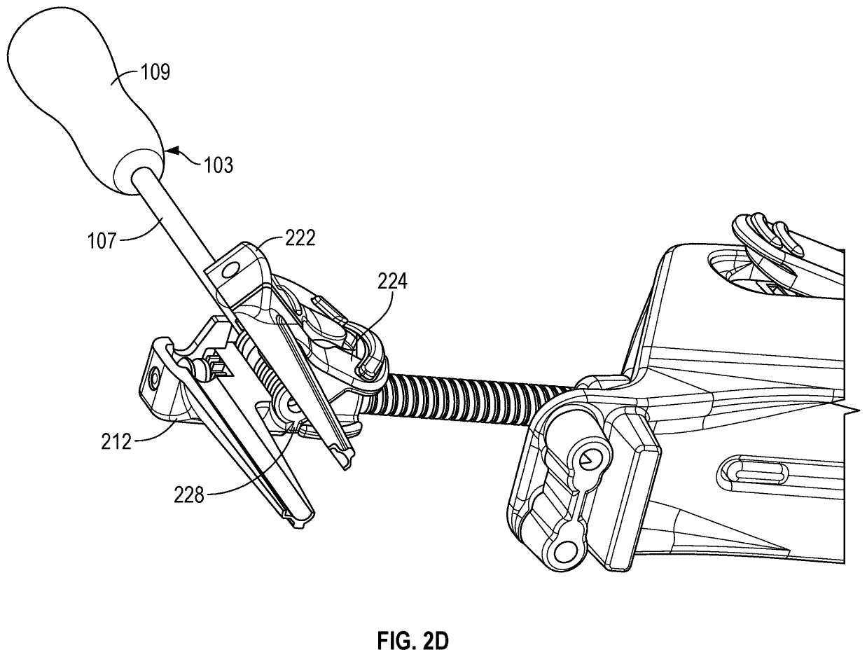

The implant drive head 101 can include a trajectory tool 103 that is removably attached to the drive head 101. In an example, the trajectory tool is insertable into a bore in the drive head 101 to assist with positioning the drive head 101 in the desired trajectory while keeping the field open. In this example, the trajectory tool is received within a trajectory bore 236 through the center of the hinge mechanism of the drive head (e.g., drive housing hinge 230). The location of the trajectory bore 236 is better illustrated in FIGS. 2A-5. In this example, the trajectory tool 103 includes a handle 109 and an elongate shaft 107 that couples into the trajectory bore 236. The surgeon can grasp the handle 109 to maneuver the drive head 101 into position without needing to grasp the drive head 101 and obstruct the surgical field or insertion location. In other examples, the trajectory tool 103 can include a laser pointer, a collimated light emitted diode (LED), or a camera. The laser pointer or collimated LED can provide a visual indication of the drive head trajectory projected onto the surgical field through the drive housing hinge 230. The camera can provide a visual display of the trajectory of the drive head 103 on a display screen.

In another embodiment, the trajectory tool 103 can interface with the channel formed by the drive head 101 (e.g., channel formed by the right drive housing 212 and bottom left drive housing 224), which also accommodates the implant during insertion. In certain examples, the right drive housing 212 and bottom left drive housing 224 can be locked into a closed position to provide a stable channel in which the trajectory tool 103 can be inserted. In this case, the trajectory tool 103 can include a camera or laser pointer, or other method of guidance, to provide additional visual guidance to the user to directly visualize the exact would-be path of the implant if the channel remained in position between use of the trajectory tool and the later engagement of the electrode. An example workflow includes the following operations: (1) the user mounts the stage 105 and drive unit 100 to the patient; (2) the users inserts the trajectory tool 103 into the channel formed by the right drive housing 212 and the bottom left drive housing 224; (3) the user enables the laser pointer (or other guiding feature) to project the vector of the drive head 101 onto the patient; (4) adjustment of the trajectory of the drive head 101 done via the trajectory tool 103 by using the trajectory tool 103 as a lever to apply forces to the drive head 103 to reorient the drive head 103, guided by the laser pointer; (5) once the desired trajectory of the drive head is achieved, the drive head 101 is opened and the trajectory tool 103 is removed; and (6) finally, the user proceeds with insertion of the implant via the drive head 101. In another example, the trajectory tool 103 can interface with the bore in the drive head via an interlocking interface, such as a threaded screw/bore. In this case the trajectory tool 103 includes an outer machine screw thread, and the trajectory bore 236 has a corresponding inner thread. The user then turns the trajectory tool 103 in the trajectory bore 236 until there is an interference fit. The interference fit could be due to the threads “running out” on the tool, i.e. the user advances the tool such that the bore thread begins to engage with a non-threaded surface. The interference fit results then in a frictional coupling, and the trajectory tool 103 provides a firm hold on the drive head for trajectory adjustment. The user can then twist in the opposite direction to disengage the interference fit and remove the trajectory tool 103 when desired. Other forms of firm, yet removable attachment are possible as well.

### Split Sheath

The implant drive head 101 includes a drive housing 101 that can house drive mechanism, an introducer sheath, and sheath components. The drive housing 101 can comprise two housing halves interconnected via a hinge (see e.g., FIGS. 2A-2D discussed in detail below). The hinge allows for opening and closing of the drive housing to engage and disengage with the elongated member or electrode of varying sizes, geometry, and diameter. The two housing halves may be designed to be completely detachable via a decouplable hinge or other temporary coupling. The drive housing may include a torsion spring. The torsion spring may apply a relative closing force to the halves such that there is a biasing force that is transferred to the components that push or pull the implant (such as drive wheels described below). The torsion spring may be designed to provide an opening force bias, which could make release or ejection of the implant from the drive housing after insertion.

### Top and Bottom Split Drive Housing

FIGS. 2A-2D illustrate, by way of example and not limitation, various views of an implant drive head 101 with a split drive housing 210. In this example, the split drive housing 210 can include a left drive housing 220 (first half drive housing) coupled together via a drive housing hinge 230 with a right drive housing 212 (second half drive housing). The left drive housing 220 can include a top left drive housing 222 and a bottom left drive housing 224, wherein the top left drive housing 202 can articulate (pivot) on the drive housing hinge 210 separately from the bottom left drive housing 224. The top left drive housing 222 can engage with an upper portion of the bottom left drive housing 224 to close both the top left drive housing 222 and bottom left drive housing 224 as a single unit. Both the top left drive housing 222 and the bottom left drive housing 224 include flanges that extend towards the goose neck 102 to enable opening via rotation around the drive housing hinge 230.

FIGS. 2A-2D illustrate, by way of example and not limitation, various views of an implant drive head with a split drive housing. In these figures, the split drive housing is illustrated including drive head with a split drive housing including a top left drive housing 202 (e.g., wheel portion) and bottom left drive housing 203 (e.g., sheath portion). In split drive housing allows for opening of just the top left drive housing 202 to enable threading the electrode or sheath directly into the sheath (lower portion) of the drive head. The top left drive housing includes the secondary wheel 204 portion of the drive head, which is responsible for advancing the implant once threaded into the drive head. In this example, both sides of the drive head (right and left) include drive wheels (e.g., both the drive wheel and the secondary wheel in the drive head are positively driven as compared to other examples where one of the wheels is an idler wheel).

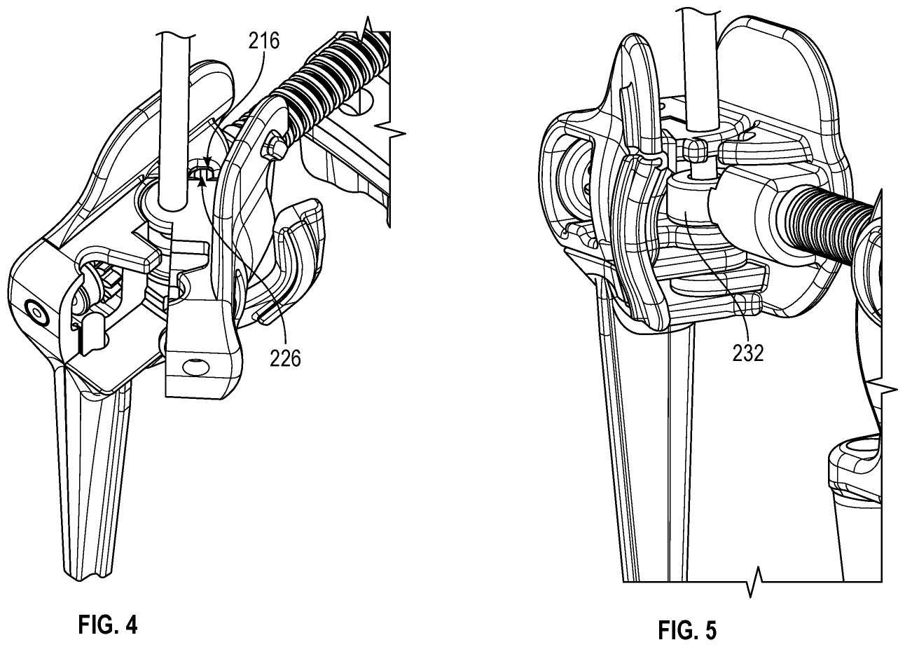

As shown in FIG. 2B, the top left drive housing 202 can be pivoted into an open position separately from the bottom left drive housing 203. In this configuration, the implant can be easily threaded into the lower drive housing (formed by the closure of the bottom left drive housing and the bottom of the right drive housing 206). As shown in detail in FIG. 4, the top left drive housing can include a locking mechanism (upper drive head lock 205) that retains the top left drive housing in an open position for implant loading. The upper drive head lock includes a locking tab on a movable portion of the top left drive housing and a corresponding recess on a fixed portion of the right drive housing. The hinge on the top left drive housing is spring loaded, to maintain traction on the implant once the drive housing is closed after loading (see FIG. 5).

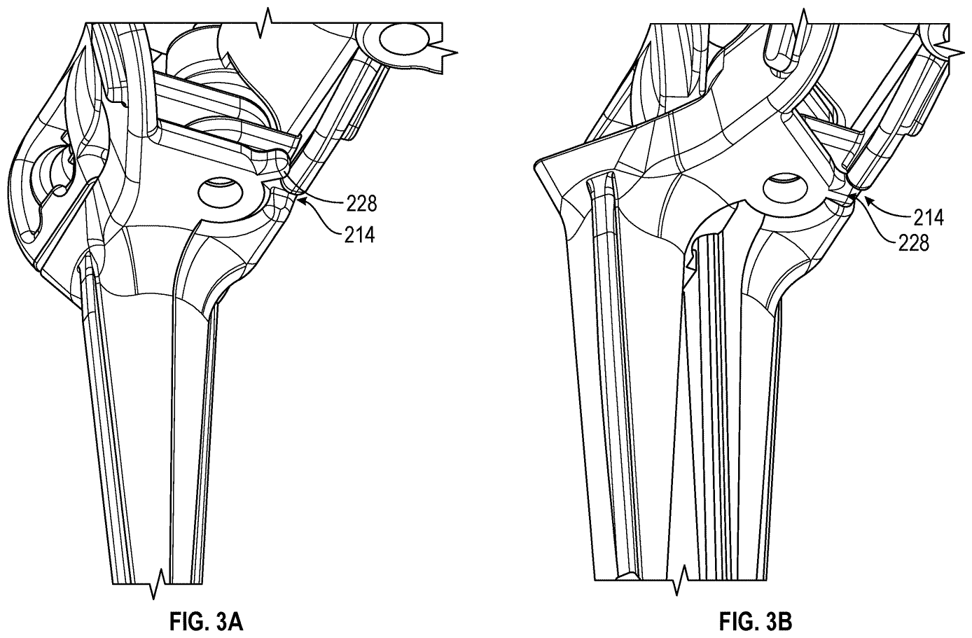

FIG. 2C illustrates the left drive housing (top left drive housing 222 and bottom left drive housing 224) in an open position. In this configuration, the drive head 101 can be easily disengaged from the implant 400 once it is positioned within the patient. The bottom left drive housing 224 includes a structure (the bottom drive head lock 228) along the upper surface that interferes with the top left drive housing 222, so that if the surgeon opens the bottom left drive housing 224 both the top and bottom portions of the left drive housing open into the position illustrated in FIG. 2C. FIGS. 2D, 3A and 3B all include illustrations of the bottom drive head lock 228. FIG. 3A illustrates the bottom drive head lock 228 in a closed position, while FIG. 3B illustrates the bottom drive head lock 228 in an open position.

FIGS. 3A-3B illustrate, by way of example and not limitation, a locking mechanism for a lower portion of a split drive housing. In this example, the bottom left drive housing 224 includes a bottom drive head lock 228. The bottom drive head lock 228 extends radially outward from the portion of the bottom left drive housing 224 that couples to the drive housing hinge 230. The bottom drive head lock 228 snaps into a lock recess 214 on a lower portion of the right drive housing 212. Once snapped into the lock recess 214, the bottom drive head lock 228 operates to hold the bottom left drive housing 224 in an open position.

FIG. 4 illustrates, by way of example and not limitation, a perspective view of an implant drive head 101 with the top left drive housing 222 or the split drive housing 210 in an open position. In this example, the top left drive housing 222 includes a top drive head lock 226. In this example, the top drive head lock 226 engages a top lock recess 216 in an upper portion of the right drive housing 212. Once the top drive head lock 226 engages the top lock recess 216 the top left drive housing 222 is held in an open position until the top drive head lock 226 is disengaged from the top lock recess 216. In this example, the top drive head lock 226 snaps into the top lock recess 216 and the top lock recess 216 provides an interference fit to keep the top drive head lock 226 engaged.

FIG. 5 illustrates, by way of example and not limitation, a rear perspective view of an implant drive head 101 with a split drive housing 210 including an upper spring hinge 232. In this example, the upper spring hinge 232 is a torsional spring.

### Drive Wheels and Torsion Spring

The drive mechanism inside the drive head 101 can include a drive wheel 104, a secondary wheel 204, and a torsion spring (upper spring hinge 232), which together form an embodiment of the drive mechanism to manipulate an elongate member/implant such as a cochlear implant or electrode. The drive wheel 104 rotates to insert or retract elongate member through an implant channel created by separate halves of the drive head 101. In an example, the secondary wheel 204 rotates to keep the elongate member aligned with the drive wheel 104. In other examples, the secondary wheel 204 can receive torque and assist in driving movement of the elongate member. Torque can be transmitted from the electric motor to the drive wheel 104 via a torque cable (such as discussed above in reference to the goose neck 102), at least a portion of which can be enclosed in the adjustable arm. The torsion spring 232 allows the drive head 101 to open and close and provides compression on the elongate member for frictional motion, and further can accommodate for varying diameters and/or shapes of the elongate member along its length. In this case, the torsional spring 232 design can be chosen such that for all compatible elongate member diameters (and therefore, for the range of resultant spring displacements), the torsional spring 232 forces remain in an acceptable range that will both avoid damaging the implant as well as provide sufficient friction to drive the implant effectively.

### Geared Drive Wheels

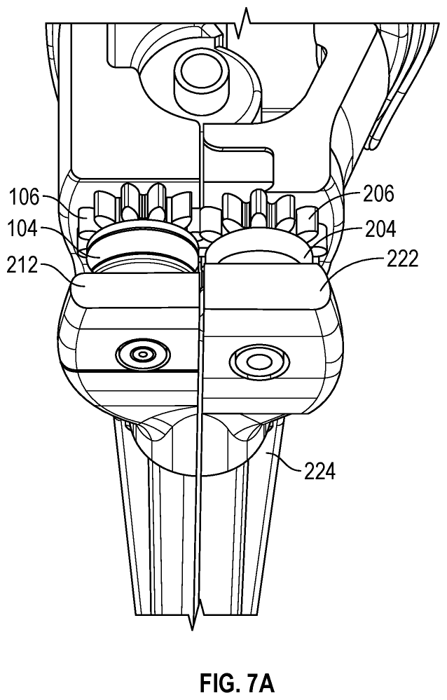

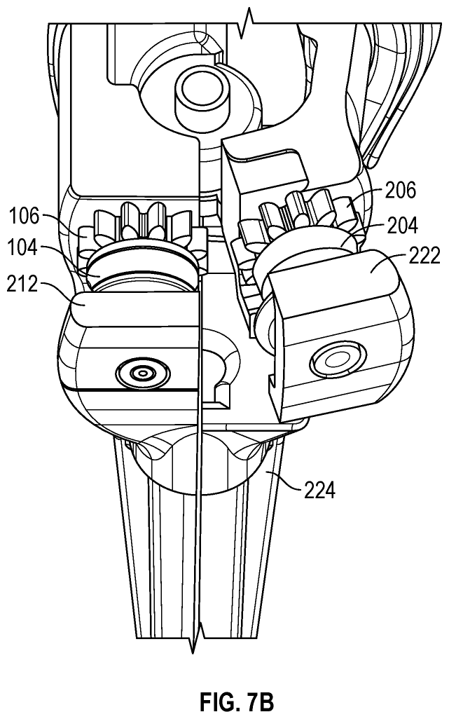

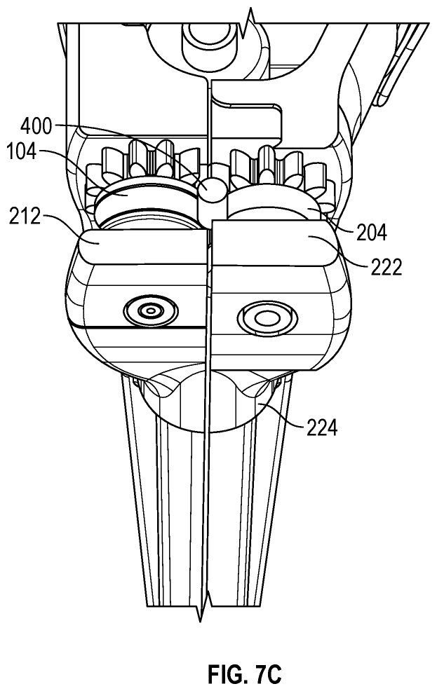

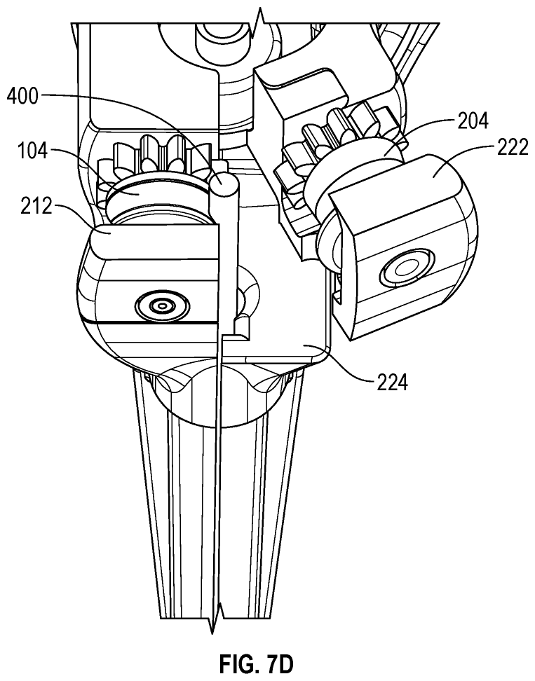

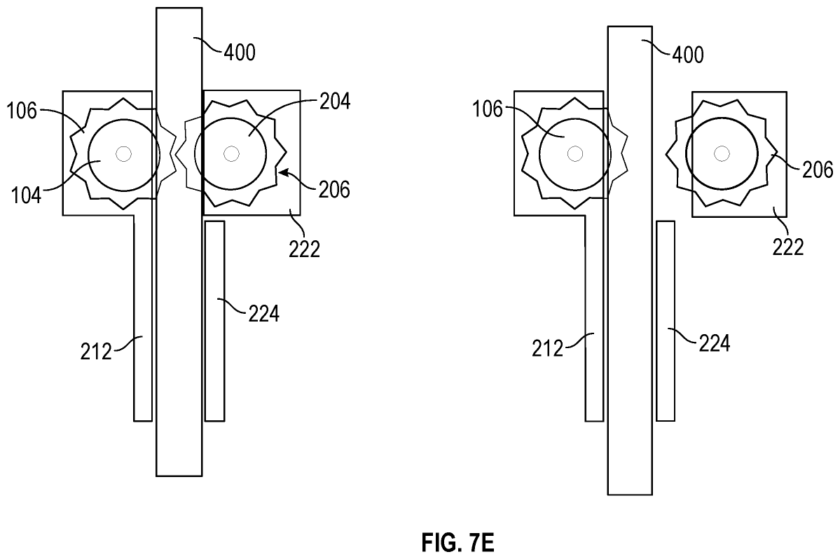

As shown in FIGS. 7A-7E, the drive wheel 104 and secondary wheel 204 may include gear features (drive gear 106 and secondary gear 206) which provide a means for some of the torque sent to the drive wheel 104 by the motor to be transferred to the secondary wheel 204 when the drive wheel and secondary wheels are close enough to be engaged. In the case of a separable drive housing (e.g., split drive hosing 210), the arrangement of the drive gear 106 and secondary gear 206 can be disengaged when the drive housing is separated. In other embodiments, the drive gear 106 and secondary gear 206 continue to engage when the housing is opened, so that the torque from both wheels provide a more balanced tangential force to the implant 400 (illustrated in FIGS. 7C and 7D). The drive gear 106 and secondary gear 206 were implemented to address an issue with engaging elongate members/implants with different diameters or elongate members/implants with varying diameters along their length. Driving both the drive wheel 104 and the secondary wheel 204 via engagement between the drive gear 106 and the secondary gear 206 allows for driven contact on opposing sides of an elongate member/implant resulting in enhanced control and increased tolerance for different diameters of implant.

### Silicone Brake

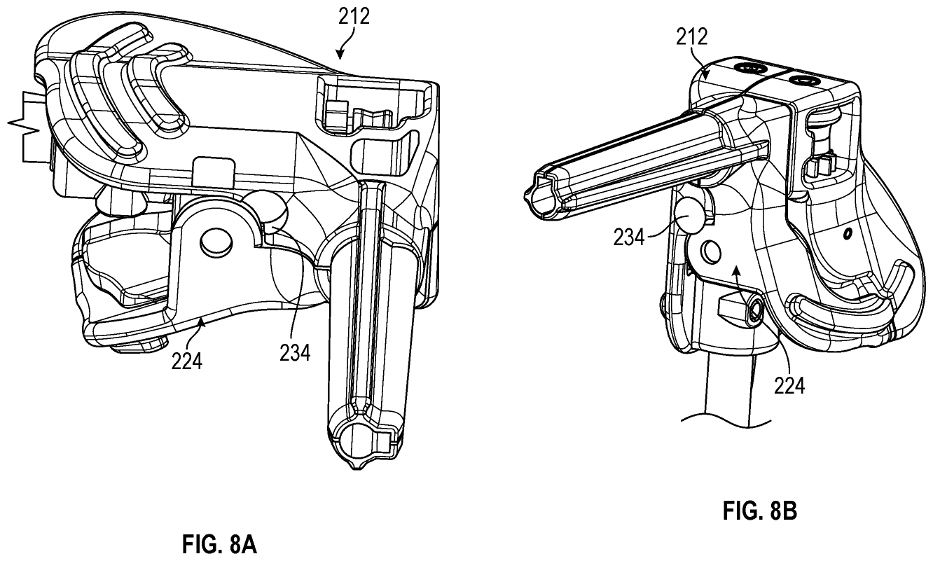

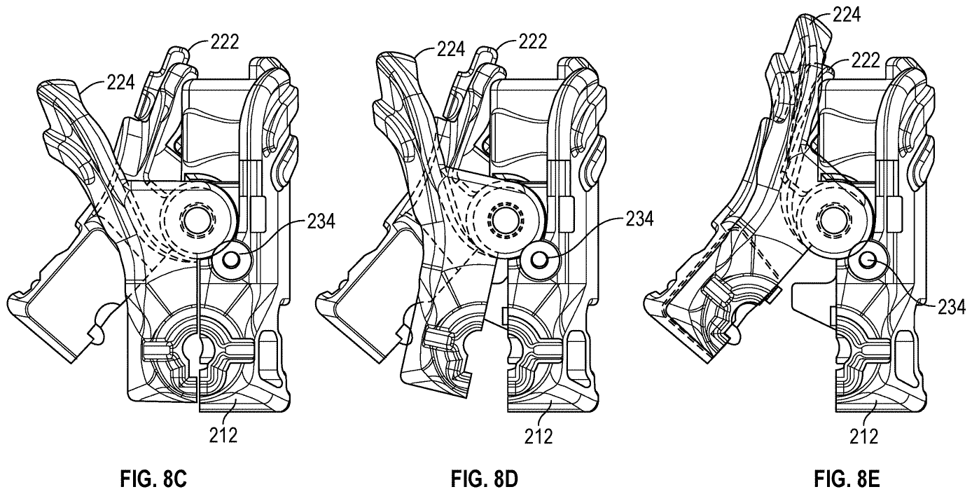

The bottom drive housing may be spring-loaded, either to an open position (thereby reliant on a bottom drive head lock to maintain the implant guiding channel) or in a closed position in order to allow for the formed channel (e.g., implant channel) that guides the implant during engagement to remain intact in lieu of a bottom drive head lock. However, a closing bias may interfere with disengagement of the implant after insertion. Therefore, in another example, a silicone brake 234 can be used along either the top left drive housing 222 or bottom left drive housing 224, in order to provide friction that tends to hold either the top left drive housing 222 or bottom left drive housing 224 in position, thereby resisting unintended forces that may partially open the drive housing, thereby potentially disengaging the implant undesirably. The silicone brake 234 may be a plug that is designed to provide an interference fit, as shown in FIGS. 8A-8E. In all these Figures, silicone brake 234 is disposed within a bottom portion of the right drive housing 212 in a position to compress against the edge of the bottom left drive housing 224. Compression of the silicone brake 234 provides friction so that the position of the bottom left drive housing 224 remains fixed in its rotation relative to the top left drive housing 222. The silicone brake 234 be made of a variety of materials, not just silicone, that can provide friction. The braking concept can also be implemented without a specific brake component, but perhaps by an interference fit between the bottom left drive housing 224 and other parts of the drive housing.

### Handheld Attachment

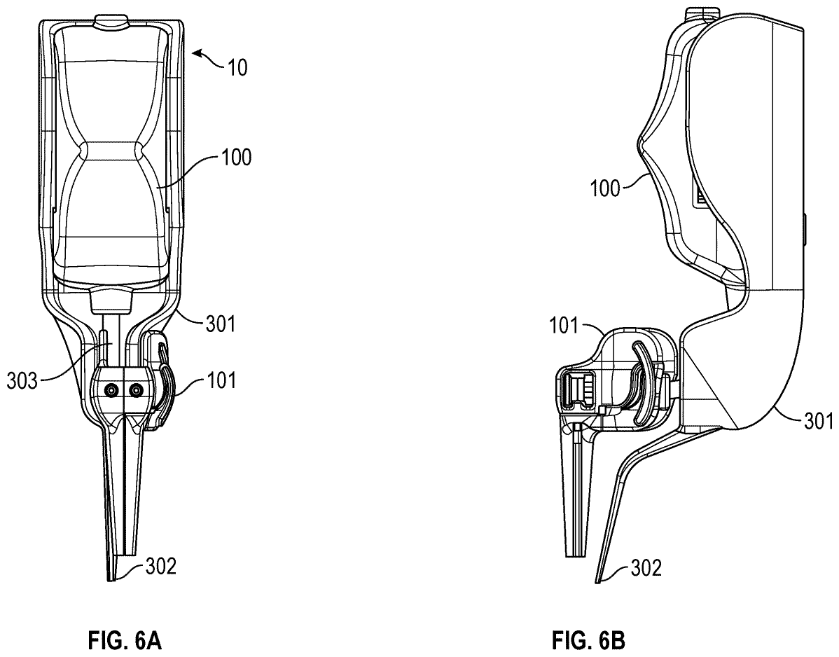

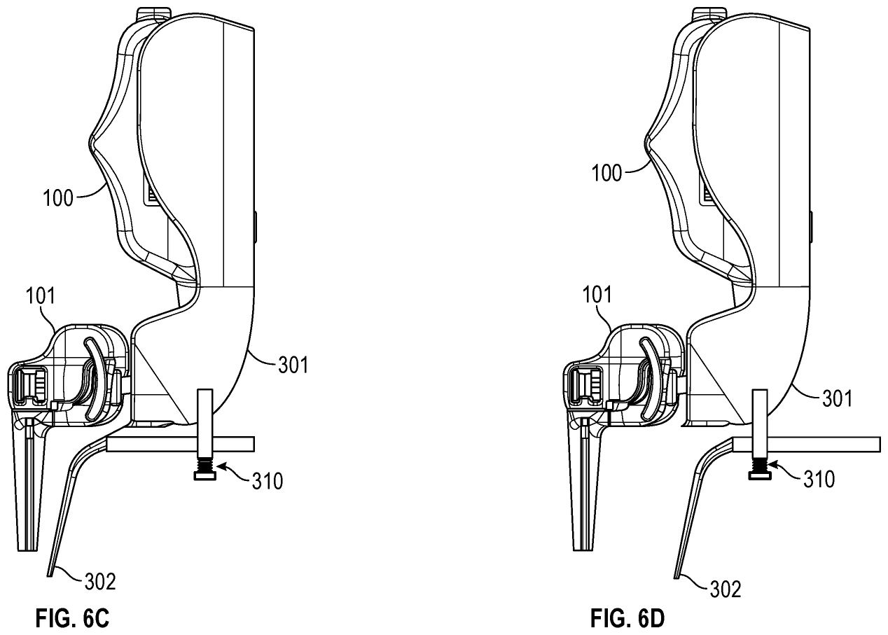

FIGS. 6A-6B illustrate, by way of example and not limitation, various views of an implant-positioning unit 10 in a handheld attachment 301. In this example, the drive unit 100 is retained within a handheld attachment 301 that does not need to be affixed to the patient. The handheld attachment 301 includes a generally U-shaped main body that receives the drive unit 100 itself. In some examples, the drive unit 100 can be shifted linearly within the main body of the handheld attachment 301. The drive head 101 and gooseneck coupling to the drive unit 100 are retained within a narrowed distal drive head section that transitions from the main body.

The handheld attachment 301 can be removably attached to the implant-positioning unit 10. This allows the handheld attachment 301 to serve as an optional accessory to the implant-positioning unit 10. It also may be permanently attached before use, either during manufacture or at some point prior to use. Permanent attachment may serve as convenient to the end user, whereas optional attachment may serve as advantageous for inventory management, packaging, or other operational consideration.

### Fulcrum Point

As illustrated in FIGS. 6A and 6B, the handheld attachment 301 may include a fulcrum point 302 in order to provide stability during insertion. The fulcrum point 302 may be a sharp tipped stylus in order to easily plant into the surface of the bone for stability, or it may be a blunt tipped stylus or post in order to allow for a secure resting place that does not damage any tissue by digging into it.

The fulcrum point 302 can be attached to the drive head 101 or drive body 100. The fulcrum point 302 attachment design may be adjustable to provide for a range of pivot locations relative to the implant-positioning unit 10. FIG. 6C shows and example of a fulcrum point 302 which is repositionable in place relative to the handheld attachment body 301. FIG. 6D shows the fulcrum point repositioned relative to the configuration in FIG. 6C. The adjustment mechanism 310 allows a user to adjust the position of the fulcrum point 302 relative to the drive head 101. In this example, the adjustment mechanism 310 is locked in place by a set-screw to hold or release a cylindrical body terminating in the fulcrum point 302. Furthermore, the fulcrum point 302 may be designed to be removable from the handheld attachment 301 as it may interfere with its use in some situations, or may be uncomfortable to the user.

### Fixed Coupling for Drive Head

In some examples, the handheld implant-positioning unit 10 includes a fixed coupling 303 between the drive head 101 and drive unit 100 as the trajectory positioning is performed relative to the fulcrum point 302 eliminating the need for an adjustable Gooseneck coupling (as shown in FIG. 1). A rigid tube cable drive channel can be used as the fixed coupling 303 in replacement of the gooseneck in other aforementioned embodiments. In the illustrative example shown in FIGS. 6A and 6B, the fixed coupling 303 holds the drive head 101 at a 90 degree angle in comparison to the longitudinal axis of the drive unit 100 and main body of the handheld attachment 301. In comparison, FIG. 1 shows the drive head 101 at a zero-degree angle relative to the drive unit 100.

The above detailed description is intended to be illustrative, and not restrictive. The scope of the disclosure should, therefore, be determined with references to the appended claims, along with the full scope of equivalents to which such claims are entitled.

### EXAMPLES

The following examples provide a non-limiting set of example systems and devices according to the present disclosure. This section provides a descriptive overview of various aspects of the invention, focusing on the various examples as claimed and disclosed. Each example corresponds to specific claims, detailing the key elements and additional features. The additional features described within the examples are optional features of the previously described system or device.
Example 1 is a system for robotically assisted manipulation of an elongate implant. This system includes a drive unit with a motor connected to a first end of a flexible drive shaft. A drive head is attached to the second end of the flexible drive shaft. The drive head comprises a first half drive housing with a first drive wheel and a second half split drive housing with a second drive wheel, both forming an implant channel. The first and second drive wheels manipulate the elongate electrode in response to rotational input from the motor via the flexible drive shaft.
Example 2 includes the subject matter of Example 1, with the additional feature that the first drive wheel includes a first drive gear, and the second drive wheel includes a second drive gear. These gears are engageable to transmit rotational forces between the first and second drive wheels.
Example 3 includes the subject matter of Example 2, wherein the drive head is coupled to the drive unit via a flexible drive shaft, and wherein a distal end of the flexible drive shaft is coupled to the first drive wheel, and the first drive gear transfers rotational force to the second drive gear.
Example 4 includes the subject matter of any one of Examples 1 to 3, with the additional feature of a housing lock. This lock is designed to secure at least one of the top drive housing and the bottom drive housing in position relative to the first half drive housing.
Example 5 includes the subject matter of Example 4, wherein the housing lock includes a top drive head lock to secure the top drive housing in position relative to the first half drive housing.
Example 6 includes the subject matter of Example 4, wherein the housing lock includes a bottom drive head lock to secure the bottom drive housing in position relative to the first half drive housing.
Example 7 includes the subject matter of any one of Examples 1 to 6, with the additional feature of a trajectory tool that can be coupled with the drive head. This tool assists with the positioning of the drive head relative to a portion of a patient's anatomy.
Example 8 includes the subject matter of Example 7, wherein the trajectory tool includes a handle and an elongate body.
Example 9 includes the subject matter of Example 8, wherein the elongate body of the trajectory tool includes a laser pointer at its distal end. This laser pointer is configured to provide a visual indication of the trajectory of the drive head.
Example 10 includes the subject matter of Example 7, wherein the drive head includes a trajectory bore to receive a distal end of the trajectory tool. In some examples, the trajectory bore includes internal threads and a portion of the distal end of the trajectory tool includes corresponding external threads.
Example 11 includes the subject matter of Example 7, wherein the trajectory tool is configured to be inserted into the implant channel.
Example 12 is an implant insertion device comprising a drive unit with a motor and a drive head configured to position an elongate implant via rotational input from the drive unit. The drive head includes a right drive housing with a right drive wheel and a left drive housing with a left drive wheel, forming an implant channel. The left drive housing includes a top drive housing including a left drive wheel and a bottom drive housing including the left half of the implant channel.
Example 13 includes the subject matter of Example 12, wherein the right drive housing includes a silicone brake designed to generate a friction fit with at least one of the top drive housing and the bottom drive housing.
Example 14 includes the subject matter of Example 12, wherein the drive head includes a drive housing hinge that pivotably couples the right drive housing with the left drive housing.
Example 15 includes the subject matter of Example 14, wherein the drive housing hinge includes an upper spring hinge that biases the top drive housing relative to the right drive housing.
Example 16 is a system for robotically assisted manipulation of an elongate implant, comprising a drive unit with a motor connected to a drive shaft, a drive head with an elongate implant manipulation mechanism, and a handheld housing for handheld operation of the drive head and drive unit. In some examples, the drive shaft is a flexible drive shaft.
Example 17 includes the subject matter of Example 16, wherein the elongate implant manipulation mechanism includes a first and a second drive wheel, which operate to manipulate the elongate electrode in response to rotational input from the motor.
Example 18 includes the subject matter of Example 16, wherein the handheld housing includes a fulcrum point that allows the system to be stabilized on a portion of anatomy adjacent to an implantation site on a patient.
Example 19 includes the subject matter of Example 18, wherein the fulcrum point is adjustable relative to the handheld housing.
Example 20 includes the subject matter of Example 16, wherein the handheld housing is configured to retain the drive unit and the drive head in a fixed position relative to the handheld housing.
Example 21 includes the subject matter of Example 20, wherein the handheld housing is configured to enable linear translation of the drive head relative to the handheld housing.

This structured approach provides a clear and comprehensive understanding of the invention's various embodiments (examples), enhancing the readability and accessibility of the patent document for technical and non-technical audiences alike.

1-21. (canceled)

22. A system for robotically assisted manipulation of an elongate implant, the system comprising:
a drive unit including a motor;
a drive head coupled to the drive unit and configured to engage and manipulate the elongate implant;
a handheld housing configured to retain both the drive unit and the drive head in a fixed position relative to the handheld housing for handheld operation; and
a fulcrum point coupled to the handheld housing and configured to provide stability during implant manipulation by engaging a portion of anatomy adjacent an implantation site.

23. The system of claim 22, wherein the fulcrum point includes a stylus configured to be planted into a surface to provide stability to the system.

24. The system of claim 23, wherein the stylus includes a sharp tip configured to penetrate the surface.

25. The system of claim 23, wherein the stylus includes a blunt tip configured to rest on tissue without penetrating the surface.

26. The system of claim 22, comprising an adjustment mechanism configured to adjust a position of the fulcrum point relative to the handheld housing.

27. The system of claim 26, wherein the adjustment mechanism includes a set-screw configured to lock the fulcrum point in a selected position.

28. The system of claim 26, wherein the adjustment mechanism is configured to lock and release the fulcrum point for repositioning.

29. The system of claim 22, wherein the fulcrum point is removable from the handheld housing.

30. The system of claim 22, wherein the handheld housing includes a U-shaped main body configured to receive the drive unit.

31. The system of claim 30, wherein the drive unit is configured to be shifted linearly within the U-shaped main body of the handheld housing.

32. The system of claim 22, comprising a fixed coupling between the drive head and the drive unit.

33. The system of claim 32, wherein the fixed coupling is configured to hold the drive head at a predetermined angle relative to a longitudinal axis of the handheld housing.

34. The system of claim 33, wherein the predetermined angle is approximately 90 degrees.

35. The system of claim 33, wherein the handheld housing is configured to enable controlled linear translation of the drive head relative to the handheld housing while maintaining the fixed coupling.

36. The system of claim 22, wherein the motor includes a hollow shaft stepper motor.

37. The system of claim 22, wherein the handheld housing comprises:
a main body configured to receive the drive unit; and
a narrowed distal section that transitions from the main body and retains the drive head.

38. A method for robotically assisted manipulation of an elongate implant, the method comprising:
engaging the elongate implant with a drive head coupled to a drive unit including a motor;
retaining both the drive unit and the drive head in a fixed position within a handheld housing for handheld operation;
stabilizing the handheld housing by engaging a fulcrum point coupled to the handheld housing with a portion of anatomy adjacent an implantation site; and
robotically manipulating the elongate implant using the drive head while maintaining handheld control.

39. The method of claim 38, further comprising adjusting a position of the fulcrum point relative to the handheld housing prior to stabilizing the handheld housing.

40. The method of claim 38, wherein stabilizing the handheld housing comprises planting a stylus of the fulcrum point into a surface to provide stability.

41. The method of claim 38, further comprising controlling the motor to provide rotational input to the drive head while maintaining the fixed position of the drive unit and drive head within the handheld housing.

42. A handheld implant-positioning device for robotically assisted manipulation of an elongate implant, comprising:
a drive unit including a motor;
a drive head coupled to the drive unit and configured to engage and manipulate an elongate member of an implant in response to rotational input from the motor;
a handheld housing configured to retain the drive unit and the drive head in a fixed position relative to the handheld housing for handheld operation; and
a fulcrum point coupled to the handheld housing and configured to provide stability during implant manipulation by engaging a portion of anatomy adjacent an implantation site.
